(19) United States
(12) Patent Application Publication
(10) Pub. No.: US 2003/0153764 A1
(43) Pub. Date: Aug. 14, 2003

(54) Process for preparing 4-substituted piperidines

(76) Inventors: Alexander Glenn Godfrey, Greenwood, IN (US); Steven Wayne Pedersen, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/148,714

(22) PCT Filed: Dec. 6, 2000
(86) PCT No.: PCT/US00/32429

(51) Int. Cl. C07D 211/44

(52) U.S. Cl. 546/217

(57) ABSTRACT

The present invention provides a process for preparing N-protected-4-substituted piperidines of formula (I) wherein Pg represents a suitable nitrogen protecting group; X represents a heterocycle, substituted heterocycle, substituted alkenyl or substituted aryl wherein the substituted heterocycle, substituted alkenyl or substituted aryl are substituted with from 1 to 3 suitable activating groups; and R¹, R², R³, R⁴, R⁵, R⁶, R⁷, and R⁸ each independently represent an alkyl, alkenyl, cycloalkyl, aryl, substituted aryl, heterocycle, or substituted heterocycle, comprising treating a compound of formula (II) wherein the substituents are defined as above, with triethylsilane and trifluoroacetic acid.

The present invention allows for the selective reduction of a tertiary alcohol on an N-protected-4-substituted piperidine without the concomitant deprotection of the protected piperidine nitrogen. The invention further allows for subsequent deprotection of the N-protected-4-substituted piperidine in one pot without isolation of the intermediate reduced compound. Thus, the present invention provides an efficient synthesis of various 4-substituted piperidines which are useful intermediates in the preparation of pharmaceuticals.

The present invention provides a process for preparing a reduced N-protected amine compound comprising reducing an N-protected amine possessing a secondary or tertiary alcohol with triethylsilane and trifluoroacetic acid

The present invention further provides a process for preparing an N-protected-4-substituted piperidine comprising treating an N-protected-4-substitued piperidine having a tertiary alcohol at the 4-position of the piperidine ring with triethylsilane and trifluoroacetic acid.

In addition, the present invention provides a process for preparing a compound of formula I:

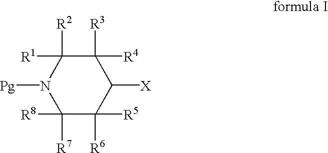

wherein Pg represents a suitable nitrogen protecting group;

X represents a heterocycle, substituted heterocycle, substituted alkenyl or substituted aryl wherein the substituted heterocycle, substituted alkenyl or substituted aryl are substituted with from 1 to 3 suitable activating groups; and

R¹, R², R³, R⁴, R⁵, R⁶, R⁷, and R⁸ each independently represent an alkyl, alkenyl, cycloalkyl, aryl, substituted aryl, heterocycle, or substituted heterocycle, comprising treating a compound of formula II:

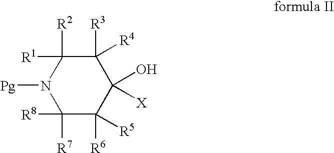

wherein the substituents are defined as above, with triethylsilane and trifluoroacetic acid.

As used herein the term “reduced N-protected amine compound” refers to a compound possessing an amine functionality that is protected with a suitable acid labile nitrogen protecting group, and any secondary or tertiary alcohol functionality has been replaced by a hydrogen atom. For example, see equation 1

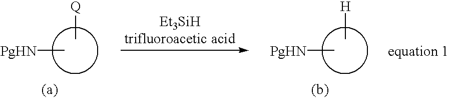

wherein

Q represents a secondary or tertiary alcohol, and the symbol;

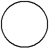

represents any suitable chemical substrate.

As used herein the term “suitable chemical substrate” refers to the remaining portion of the entire compound (a) or (b) above which does not include substituents “Q” or “PgHN—”, and which will not react with triethylsilane in the presence of trifluoroacetic acid unless such additional reaction is desired.

As used herein the piperidine of formulas I, Ia, and II have the following numbering system for the piperidine ring using formula I as an example:

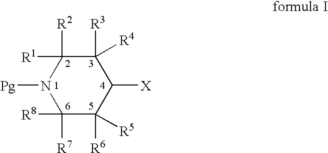

As used herein, the terms “Me”, “Et”, “Pr”, “iPr”, “Bu” and “t-Bu” refer to methyl, ethyl, propyl, isopropyl, butyl and tert-butyl, respectively.

As used herein, the terms “Halo”, “Halide” or “Hal” refer to a chlorine, bromine, iodine or fluorine atom, unless otherwise specified herein.

As used herein the term “alkyl” refers to a straight or branched, monovalent, saturated aliphatic chain. It is understood that the term “alkyl” includes within its definition the terms “C₁-C₂₀ alkyl”, “C₁-C₁₀ alkyl”, “C₁-C₆ alkyl”, and “C₁-C₄ alkyl”.

As used herein the term “C₁-C₄ alkyl” refers to a straight or branched, monovalent, saturated aliphatic chain of 1 to 4 carbon atoms and includes, but is not limited to methyl, ethyl, n-propyl, isopropyl, n-butyl, isobutyl and the like.

As used herein the term “C₁-C₆ alkyl” refers to a straight or branched, monovalent, saturated aliphatic chain of 1 to 6 carbon atoms and includes, but is not limited to methyl, ethyl, n-propyl, isopropyl, n-butyl, isobutyl, t-butyl, n-pentyl, n-hexyl, and the like.

As used herein the term “C₁-C₁₀ alkyl” refers to a straight or branched, monovalent, saturated aliphatic chain of 1 to 10 carbon atoms and includes, but is not limited to methyl, ethyl, propyl, isopropyl, n-butyl, isobutyl, tertiary butyl, pentyl, isopentyl, hexyl, 2,3-dimethyl-2-butyl, heptyl, 2,2-dimethyl-3-pentyl, 2-methyl-2-hexyl, octyl, 4-methyl-3-heptyl and the like.

As used herein the term “C₁-C₂₀ alkyl” refers to a straight or branched, monovalent, saturated aliphatic chain of 1 to 20 carbon atoms and includes, but is not limited to, methyl, ethyl, propyl, isopropyl, butyl, isobutyl, t-butyl, pentyl, isopentyl, hexyl, 3-methylpentyl, 2-ethylbutyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-nonadecyl, n-eicosyl and the like.

As used herein the term “C₁-C₆ alkoxy” refers to a straight or branched alkyl chain having from one to six carbon atoms attached to an oxygen atom. Typical C₁-C₆ alkoxy groups include methoxy, ethoxy, propoxy, isopropoxy, butoxy, t-butoxy, pentoxy and the like. The term “C₁-C₆ alkoxy” includes within its definition the term “C₁-C₄ alkoxy”.

As used herein the term “halo(C₁-C₆)alkyl” refers to a straight or branched alkyl chain having from one to six carbon atoms with 1, 2 or 3 halogen atoms attached to it. Typical halo(C₁-C₆)alkyl groups include chloromethyl, 2-bromoethyl, 1-chloroisopropyl, 3-fluoropropyl, 2,3-dibromobutyl, 3-chloroisobutyl, iodo-t-butyl, trifluoromethyl and the like. The term “halo(C₁-C₆)alkyl” includes within its definition the term “halo(C₁-C₄)alkyl”.

As used herein the term “cycloalkyl” refers to a saturated hydrocarbon ring structure. It is understood that the term “cycloalkyl” includes within its definition the term “C₃-C₈ cycloalkyl”. Typical C₃-C₈ cycloalkyl groups include cyclopropyl, cyclobutyl, cyclopentyl, cyclohexyl, cycloheptyl, cyclooctyl, and the like.

As used herein the term “alkenyl” refers to a straight or branched, monovalent, unsaturated aliphatic chain. It is understood that the term “alkenyl” includes within its definition the term “C₂-C₆ alkenyl”. Typical C₂-C₆ alkenyl groups include ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein the term “aryl” refers to a monovalent carbocyclic group containing one or more fused or non-fused phenyl rings and includes, for example, phenyl, 1- or 2-naphthyl, 1,2-dihydronaphthyl, 1,2,3,4-tetrahydronaphthyl, and the like.

As used herein the term “heterocycle” refers to a stable 5- to 7-membered monocyclic or 7- to 10-membered bicyclic heterocyclic ring which is saturated or unsaturated, and consists of carbon atoms and from one to three heteroatoms selected from the group consisting of nitrogen, oxygen or sulfur, and wherein the nitrogen and sulfur heteroatoms may optionally be oxidized, and the nitrogen heteroatom may optionally be quaternized and including a bicyclic group in which any of the above-defined heterocyclic rings is fused to a benzene ring. The heterocyclic ring may be attached at any heteroatom or carbon atom which affords a stable structure.

Examples of such heterocycles include piperidinyl, piperazinyl, azepinyl, pyrrolyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazolyl, benzopyranyl, benzothiazolyl, benzoazolyl, furyl, tetrahydrofuryl, tetrahydropyranyl, thienyl, benzothienyl, thiamorpholinyl, thiamorpholinyl-sulfoxide, thiamorpholinylsulfone, oxadiazolyl, triazolyl, tetrahydroquinolinyl, tetrahydrisoquinolinyl, and the like.

Unless otherwise specified, the term “substituted” as used in the term “substituted alkenyl”, “substituted aryl” and “substituted heterocycle” signifies that one or more (for example one or two) substituents may be present on the alkenyl, aryl or heterocycle. Examples of substituents which may be present are H, F, Cl, Br, I, OH, C₁-C₆ alkyl, C₁-C₆ alkoxy, halo(C₁-C₆)alkyl, phenyl, NO₂. NH₂, CN, or phenyl substituted with from 1 to 3 substituents selected from the group consisting of F, Cl, Br, I, OH, C₁-C₆ alkyl, C₁-C₆ alkoxy, halo(C₁-C₆)alkyl, phenyl, NO₂, NH₂, and CN.

When X represents substituted aryl or substituted heterocycle, it is preferred that the substituted aryl or substituted heterocycle do not contain an electron withdrawing substituent.

More specifically, when X represents a substituted heterocycle, substituted alkenyl or substituted aryl, the substituted heterocycle, substituted alkenyl or substituted aryl is substituted with from 1 to 3 suitable activating groups, such as substituents which are electron donating. Examples of suitable activating groups are OH, C₁-C₆ alkyl, C₁-C₆ alkoxy, phenyl, NH₂, NH(C₁-C₆ alkyl), N(C₁-C₆ alkyl)₂, and the like.

The designation “ [symbol] ” refers to a bond that protrudes forward out of the plane of the page.

The designation “ [symbol] ” refers to a bond that protrudes backward out of the plane of the page.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds of formulas I or Ia can be resolved by one of ordinary skill in the art using standard techniques well known in the art, such as those described by J. Jacques, et al., “Enantiomers, Racemates, and Resolutions”, John Wiley and Sons, Inc., 1981.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The specific stereoisomers and enantiomers of compounds of formula (I) can be prepared by one of ordinary skill in the art utilizing well known techniques and processes, such as those disclosed by Eliel and Wilen, “Stereochemistry of Organic Compounds”, John Wiley & Sons, Inc., 1994, Chapter 7, Separation of Stereoisomers. Resolution. Racemization, and by Collet and Wilen, “Enantiomers, Racemates, and Resolutions”, John Wiley & Sons, Inc., 1981. For example, the specific stereoisomers and enantiomers can be prepared by stereospecific syntheses using enantiomerically and geometrically pure, or enantiomerically or geometrically enriched starting materials. In addition, the specific stereoisomers and enantiomers can be resolved and recovered by techniques such as chromatography on chiral stationary phases, enzymatic resolution or fractional recrystallization of addition salts formed by reagents used for that purpose.

As used herein, “Pg” refers to suitable nitrogen protecting group such as an acid labile nitrogen protecting group. Examples of an acid labile nitrogen protecting group as used herein refers to those groups intended to protect or block the nitrogen group against undesirable reactions during synthetic procedures, whereby the basicity of the amine functionality is significantly reduced. Choice of the suitable nitrogen protecting group used will depend upon the conditions that will be employed in subsequent reaction steps wherein protection is required, and is well within the knowledge of one of ordinary skill in the art. Commonly used nitrogen protecting groups are disclosed in Greene, “Protective Groups In Organic Synthesis,” (John Wiley & Sons, New York (1981)). Acid labile carbamates are most suitable such as the following derivatives: 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dlimethoxybenzyloxycarbonyl, 3,4,5-trimethoxybenzyloxycarbonyl, α,α-dimethyl-3,5-dimethoxybenzyloxycarbonyl, benzhydryloxycarbonyl, t-butyloxycarbonyl, allyloxycarbonyl, 2-methyl-but-2-enyloxycarbonyl, 2,3-dimethyl-but-2-enyloxycarbonyl. Preferred acid labile nitrogen protecting groups are, t-butyloxycarbonyl (Boc) and 3,4,5-trimethoxybenzyloxycarbonyl. t-Butyloxycarbonyl (Boc) is the most preferred suitable nitrogen protecting group.

The compounds of formulas I and Ia can be prepared by following the procedures as set forth in Scheme I. All substituents, unless otherwise indicated, are previously defined. The reagents and starting materials are readily available to one of ordinary skill in the art.

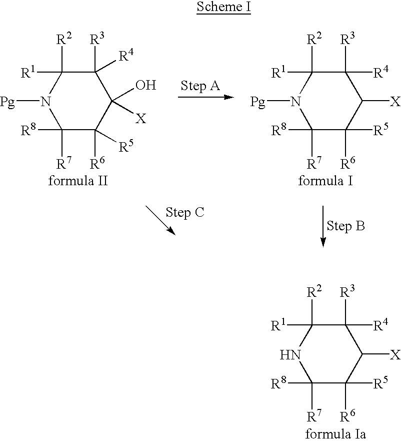

In Scheme I, step A, the compound of formula II is dissolved in a suitable organic solvent, such as methylene chloride and treated with about 4 equivalents to about 5 equivalents of triethylsilane, preferably about 5 equivalents of triethylsilane. The solution is cooled to about −40° C. to about −25° C., preferably about −30° C. The solution is then treated slowly with about 4 equivalents to about 5 equivalents of trifluoroacetic acid, preferably about 5 equivalents of trifluoroacetic acid. The reaction is then allowed to warm to about 0-5° C. over about 30 minutes to about one hour with stirring. The product, formula I, is then isolated and purified by standard techniques well known in the art, such as extraction techniques and chromatography. For example, the reaction mixture is treated with ice water and aqueous sodium hydroxide with stirring. The layers are separated and the aqueous layer is extracted with methylene chloride. The organic layer and extracts are combined, dried over anhydrous sodium sulfate, filtered, and concentrated under vacuum to provide the crude compound of formula I. This crude material can then be purified by flash chromatography on silica gel with a suitable eluent, such as ethyl acetate/hexanes.

In Scheme I, step B, the compound of formula I can be deprotedted under standard conditions well known in the art, such as those disclosed in Greene, “Protective Groups In Organic Synthesis,” (John Wiley & Sons, New York (1981)), to provide the compound of formula la. For example, the compound of formula I is dissolved in a suitable organic solvent, such as methylene chloride, the solution is cooled to about 0° C., and treated with an excess of trifluoroacetic acid. The reaction mixture is allowed to stir for about 2 to 8 hours and the product, formula la, is then isolated and purified by using standard techniques well known in the art. For example, the reaction is treated with ice water and aqueous sodium hydroxide with stirring. The layers are separated and the aqueous layer is extracted with methylene chloride. The organic layer and organic extracts are combined, dried over anhydrous sodium sulfate, filtered, and concentrated under vacuum to provide the crude material of formula Ia. This crude material can then be purified by flash chromatography on silica gel with a suitable eluent, such as ethyl acetate/hexanes.

In Scheme I, step C, the compound of formula I is dissolved in a suitable organic solvent, such as methylene chloride and treated with about 4 equivalents to about 5 equivalents of triethylsilane, preferably about 5 equivalents of triethylsilane. The solution is cooled to about −40° C. to about −25° C., preferably about −30° C. The solution is then treated slowly with about 4 equivalents to about 5 equivalents of trifluoroacetic acid, preferably about 5 equivalents of trifluoroacetic acid. The reaction is then allowed to warm to about 0-5° C. over about 30 minutes to about one hour with stirring, and an additional 4 to 5 equivalents of trifluoroacetic acid is added. The reaction is then allowed to warm to room temperature, and the product, formula Ia, is isolated and purified by standard techniques well known in the art, such as extraction techniques and chromatography. For example, the reaction mixture is treated with ice water and aqueous sodium hydroxide with stirring. The layers are separated and the aqueous layer is extracted with methylene chloride. The organic layer and extracts are combined, dried over anhydrous sodium sulfate, filtered, and concentrated under vacuum to provide the crude compound of formula Ia. This crude material can then be purified by flash chromatography on silica gel with a suitable eluent, such as ethyl acetate/hexanes.

The following examples are illustrative only and represent typical syntheses of the compounds of formula I as described generally above. The reagents and starting materials are readily available to one of ordinary skill in the art. As used herein, the following terms have the meanings indicated: “eq” or “equiv.” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to parts per million down-field from tetramethylsilane; “THF” refers to tetrahydrofuran; “DMF” refers to N,N-dimethyiformamide; “DMSO” refers to methyl sulfoxide; “LDA” refers to lithium diisopropylamide; “aq” refers to aqueous; “EtOAc” refers to ethyl acetate; “iPrOAc” refers to isopropyl acetate; “MeOH” refers to methanol; “MTBE” refers to tert-butyl methyl ether, and “RT” refers to room temperature.

### Preparation 1

Preparation of 5-Bromo-3-methyl-1-trimethylsilylbenzo[b]thiophene.

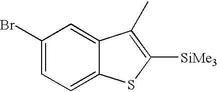

A solution of 5-bromo-3-methylbenzo[b]thiophene (149.1 g, 0.66 mole) in THF (1.4 L) under nitrogen was cooled to −78° C. and trimethylsilyl chloride (163 mL, 1.3 mole, 2 eq) was added dropwise. Lithium diisopropylamide (625 mL, 1.2 mole, 2 eq, 2.0 M solution in THF, heptane, ethylbenzene) was added and the mixture was stirred for 4 h. The solution was poured into a mixture of methyl tert-butylether and H₂O (3 L each). The layers were separated and the organic layer was extracted with 1 N HCl (2 L), then H₂O (2 L) and dried (Na₂SO₄). The solvent was removed by rotary evaporation to afford 237.3 g of crude product. The crude material was slurried in EtOH (400 mL) to afford 5-bromo-3-methyl-1-trimethylsilylbenzo[b]thiophene as a white granular solid (152.7 g, 78%, 3 crops).

mp64-67° C. IR (KBr) 1252, 1245, 841 cm⁻¹;

¹H NMR (300 MHz, CDCl₃) δ 7.851 (d, 1, J=1.8 Hz), 7.69 (d, 1, J=8.5 Hz), 7.41 (dd, 1, J=8.5, 1.8 Hz), 2.48 (s, 3,), 0.42 (d, 9, J=3.4 Hz). ¹³CNMR (75 MHz, CDCl₃) δ 143.6, 141.3, 137.9, 132.2, 126.9, 124.4, 123.4, 117.8, 14.4, 0.14. MS (FD) m/z 298 (M+). Anal. Calcd for C₁₀H₁₅BrSSi: C, 48.16; H, 5.05. Found: C, 48.19; H, 4.98.

### Preparation 2

Preparation of 1-(t-Butyloxycarbonyl)-4-(3-methylbenzo[b]thiophen-5-yl)-piperidin-4-ol.

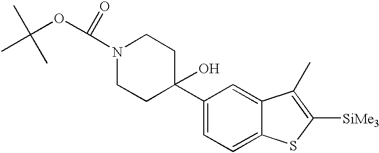

To a solution of 5-bromo-3-methyl-1-trimethylsilylbenzo[b]thiophene (211.7 g, 707 mmol) in THF (1 L) cooled to −78° C. under nitrogen was added n-BuLi (311 mL, 2.5 M solution in hexanes, 778 mmol) dropwise. After 30 min, N-Boc-piperidone (155.1 g, 778 mmol) in THF (816 mL) was added. After 2 hr, the mixture was poured into H₂O and methyl tert-butylether (2 L each). The layers were separated and the organic layer was washed with 1 N HCl (2.1 L), then H₂O (2.1 L) and dried (Na₂SO₄). The solvent was removed with a rotary evaporator to afford 348 g of crude 1-(t-butyloxycarbonyl)-4-(3-methyl-1-trimethylsilylbenzo[b]thiophen-5-yl)-piperidin-4-ol. Hexane (700 mL) was added to the crude product. After stirring overnight, the precipitate was filtered, washed with hexane, and dried in a vacuum oven for 2 hr to give 246.3 g (83%) of 1-(t-butyloxycarbonyl)4-(3-methyl-1-trimethylsilylbenzolblthiophen-5-yl)-piperidin-4-ol as a white powder. mp 141-145° C. IR (CHCl₃) 3595, 1680 cm⁻¹.

¹H NMR (300 MHz, CDCl₃), δ 7.83 (d, 1, J=8.0 Hz), 7.81 (s, 1), 7.43 (dd, 1, J=8.2, 1.8 Hz), 4.06 (br s, 2), 3.28 (t, 2, J=12.2 Hz), 2.51 (s, 3), 2.09 (br s, 2), 1.79 (d, 2, J=12.2 Hz), 1.70 (s, 1), 1.49 (s, 9), 0.40 (s, 9). ¹³C NMR (75 MHz, DMSO) δ 154.2, 145.9, 141.5, 140.3, 139.1, 134.1, 122.2, 121.8, 117.7, 78.6, 70.2, 38.0, 28.3, 14.4, 0.00. MS (FD) m/z 418 (M−1). Anal. Calcd for C₂₂H₃₃NO₃SSi: C, 62.97; H, 7.93; N, 3.34. Found: C, 63.28; H, 8.04; N, 3.44.

### Preparation 3

Preparation of (±) N-t-Butoxycarbonyl-4-hydroxy-4-(6-methoxybenzo[b]thiophen-2-yl)-2-methylpiperidine.

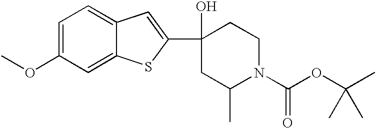

To a solution of 6-methoxybenzo[b]thiophene (5.0 g, 30.4 mmol) in dry THF (60 mL) at −78° C. was added 1.6 M n-BuLi in hexanes (20.9 mL, 33.44 mmol). The solution was stirred at −78° C. for 90 min. The N-t-butoxycarbonyl-2-methyl-4-piperidone (3.89 g, 18.24 mmol) dissolved in THF (40 mL) was added via a cannula at −78° C. The reaction mixture was stirred at −78° C. for 3 h. The reaction was then quenched with 75 mL of saturated aqueous NaCl solution. The mixture was extracted with (1×75 mL, 2×125 mL) EtOAc. The combined organic layers were dried over CaCl₂ and filtered. The filtrate was concentrated and purified by medium pressure chromatography (30% Et₂O/hexanes) to give the intermediate title compound as a yellow foam (1.798 g, 26%). IR (KBr) 3009, 2978 cm⁻¹. Ion Spray MS 378 (M+H)⁺; 436 (M+CH₃COO⁻)⁻.

### Preparation 4

Preparation of N-t-Butoxycarbonyl-4-hydroxy-4-(6-methylbenzo[b]thiophen-2-yl)piperidine.

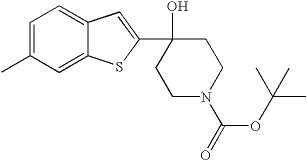

To a solution of 6-methylbenzo[b]thiophene (1.25 g, 8.43 mmol) in dry THF (20 mL) at −78° C. was added 1.6 M n-BuLi in hexanes (6.32 mL, 10.1 mmol). The solution was stirred at −78° C. for 40 min. 1-t-Butoxycarbonyl-4-piperidone (1.84 g, 9.27 mmol) dissolved in THF (10 mL) was added via a cannula at −78° C. The reaction mixture was stirred at −78° C. for 3 h. The reaction was then quenched with 50 mL of water. The mixture was extracted (3×75 mL) with EtOAc. The combined organic layers were dried over MgSO₄ and filtered. The filtrate was concentrated to an oil and allowed to stand 3 days in which time the material crystallized. The crystals were rinsed with a mixture of EtOAc/hexanes to give the intermediate title compound as yellow crystals (2.13 g, 72.6%). IR (KBr) 1681, 1429, 1246 cm⁻¹. FD+MS 347.0 (M).

### Preparation 5

Preparation of N-t-Butoxycarbonyl-4-hvdroxy-2-methyl-4-(6-methylbenzo[b]thiophen-2-yl)piperidine.

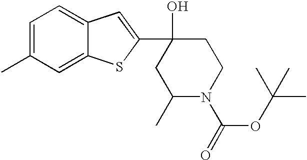

To a solution of 6-methylbenzo[b]thiophene (6.11 g, 41.21 mmol) in dry THF (90 mL) at −78° C. was added 1.6 M n-BuLi in hexanes (30.9 mL, 49.4 mmol). The solution was stirred at −78° C. for 40 min. The N-t-butoxycarbonyl-2-methyl4-piperdone (5.27 g, 24.7 mmol) dissolved in THF (47 mL) was added via a cannula at −78° C. The reaction mixture was stirred at −78° C. for 3 h. The reaction was then quenched with 200 mL of water. The mixture was extracted (3×200 mL) with EtOAc. The combined organic layers were dried over MgSO₄ and filtered. The filtrate was concentrated and run through a column of silica gel (17% EtOAc/hexanes) to give the intermediate title compound with some unreacted N-t-butoxycarbonyl-2-methyl-4-piperidone as an orange oil (6.75 g, 45%). IR (KBr) 1680, 1418, 1366, 1158 cm⁻¹. Ion Spray MS 420 (M+CH₃COO⁻)⁻.

### Preparation 6

Preparation of N-t-Butoxycarbonyl-4-(8-methoxynaphth-2-yl)-4-piperidinol.

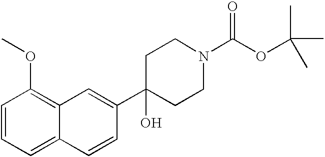

To a solution of 7-bromo-1-methoxynaphthalene (1.50 g, 6.33 mmol) in dry THF (30 mL) at −78° C. was added 1.6 M n-BuLi in hexanes (4.35 mL, 6.96 mmol). The solution was stirred at −78° C. for 15 min. N-t-Butoxycarbonyl-4-piperidone (1.51 g, 7.59 mmol) dissolved in THF (10 mL) was added via a cannula at −78° C. The reaction mixture was stirred at −78° C. for 2.5 h. The reaction was then quenched with 30 mL of saturated aqueous NH₄Cl solution. The mixture was extracted (2×150 mL) with EtOAc. The combined organic layers were then dried over MgSO₄ and filtered. The filtrate was concentrated and purified by silica gel chromatography (25% EtOAc/hexanes) to give the intermediate title compound as a white foam (1.42 g, 63%). IR (CHCl₃) 3350 (br), 1681 cm⁻¹. Ion Spray MS 358 (M+H)⁺; 240 (M-117(-(Boc+H₂O)))⁺; 430 (M+CH₃COO⁻)⁻. ¹HNMR (CDCl₃) δ 8.31 (d, J=2.0 Hz, 1H), 779 (d, J=8.3 Hz, 1H), 7.60 (dd, J=8.8, 2.0 Hz, 1H), 7.34-7.40 (m, 2H), 6.81 (dd, J=7.1, 2.0 Hz, 1H), 4.03-4.06 (br m, 2H), 3.99 (s, 3H), 3.29 (br dt, J=13.0, 2.4 Hz, 2H), 2.12 (dt, J=13.0, 4.9 Hz, 2H), 1.79-1.83 (br m, 2H), 1.61 (br s, 1H), 1.48 (s, 9H).

### Preparation 7

Preparation of 1-(t-Butyloxycarbonyl)-4-(6-methoxynaphth-2-yl)-2-methylpiperidin-4-ol.

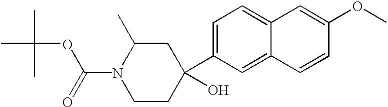

Scheme I, step A: To a solution of 2-bromo-6-methoxynaphthalene (13.009 g, 54.9 mmol) in tetrahydrofuran (400 mL) at −78° C. was added dropwise t-butyllithium (71.0 mL, 0.121 mol). After 30 minutes at −78° C., a solution of 1-(t-butyloxycarbonyl)-2-methyl-4-piperidone (12.87 g, 60.4 mmol) in tetrahydrofuran (50 mL) was added dropwise. The mixture was stirred at −78° C. for 4 hours and then diluted with saturated ammonium chloride and extracted 3 times with ethyl acetate. The combined organic layers were dried over sodium sulfate, filtered and evaporated. The residue was purified by silica gel chromatography (dichloromethane/2% methanol in dichloromethane gradient eluent) to give 5.81 g (29%) of the title compound as a yellow oil. FDMS m/e=362 (M⁺+1).

### Preparation 8

Preparation of N-t-Butoxycarbonyl-4-(4-ethylbenzo[b]thiophen-2-yl)-2-methyl-4-piperidinol.

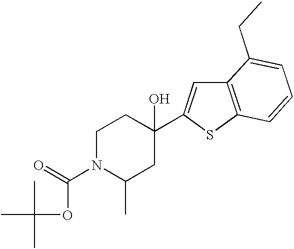

Preparation of (±)-N,N-Dimethyl-2-(2-ethylphenyl)-2-hydroxythioacetamide.

A solution of 30.11 g of 1-bromo-2-ethylbenzene in ca. 500 mL of freshly distilled THF was treated with 112 mL of 1.6 M n-BuLi in hexanes at −78° C. over a period of ca. 3 h. To this was added 15 mL of anhydrous DMF, and the mixture was stirred at −78° C. for 30 min. The cold bath was removed, and the reaction was quenched with ca. 300 mL of saturated aqueous NH₄Cl. The layers were 2S separated, and the organic layer was washed with ca. 300 mL of brine. The aqueous layers were back extracted with 2×500 mL of EtOAc. Combined organic layers were dried over MgSO₄, concentrated, and dried under house vacuum to yield 20.89 g (96%) of fairly clean crude 2-ethylbenzaldehyde.

To 290 mL of diisopropylamide in ca. 500 mL of freshly distilled THF at −70° C. was added 117 mL of 1.6 M n-BuLi in hexanes, and the yellow solution was stirred at −70° C. for 20 min, for 15 min without the cold bath, then re-cooled to −73° C. To this was added a pre-cooled (−70° C.) mixture of 20.89 g of the crude benzaldehyde and 16 mL of N,N-dimethylthioformamide in 70 mL of freshly distilled THF via a cannula over 15 min. The reddish clear solution was stirred at −75° C. for 45 min, then the cold bath was removed, and the mixture was stirred for another 30 min. The reaction was quenched with ca. 300 mL of saturated aqueous NH₄Cl, and the layers were separated. The aqueous layer was extracted with 3×500 mL of EtOAc. The organic layers were washed with ca. 300 mL of brine, combined, dried over MgSO₄, and concentrated. The residue was crystallized from EtOAc-hexanes to afford 25.20 g (73%) of yellowish crystalline solid. IR (CHCl₃) ˜3200 (br), 3009, 1529, 1387 cm⁻¹. mp 104-105° C. Ion Spray MS 223.9 (M+H)⁺. C₁₂H₁₇NOS

| analysis: | Calculated | found |
|---|---|---|
| C | 64.54 | 64.70 |
| H | 7.67 | 7.73 |
| N | 6.27 | 6.31 |

Preparation of 4-Ethyl-2-(N,N-dimethylamino)benzo[b]thiophene.

N,N-Dimethyl-2-(2-ethylphenyl)-2-hydroxythioacetamide (25.1 g, 112 mmol) was dissolved in Eaton's reagent (7.5% w/w P₂O₅/MeSO₃H) (330 mL). The reaction mixture was heated to 80° C. and stirred for 1 h. The reaction mixture was then cooled to room temperature and stirred for an additional 1.5 h. The reaction was quenched by pouring the reaction mixture slowly into cooled (0° C.) 5.0 N NaOH (1.60 L). The mixture was extracted with EtOAc (2×1.50 L). The combined organic layers were then dried over MgSO₄ and concentrated to yield the title benzo[b]thiophene (21.66 g, 94% crude yield) as a red oil. EIMS 205 M⁺; 190 (M-15)⁺(base peak).

¹HNMR (CDCl₃) δ 7.42 (d, J=7.8 Hz, 1H), 7.04 (d, J=7.3 Hz, 1H), 6.98 (t, J=7.6 Hz, 1H), 5.98 (s, 1H), 3.01 (s, 6H), 2.82 (q, J=7.8 Hz, 2H), 1.30 (t, J=7.8 Hz, 3H).

Preparation of 4-Etylthianapthen-2-one.

4-ethyl-2-dimethylaminobenzo[b]thiophene (11.10 g, 54.0 mmol) was dissolved in a 1:1 mixture of THF/1.0 N HCl (380 mL). The biphasic mixture was stirred vigorously and heated at reflux for 3 h 15 min. The reaction mixture was then cooled to room temperature and the layers were separated. The aqueous layer was extracted with EtOAc (2×400 mL). The combined organic layers were dried over MgSO₄ and concentrated to give 4-ethylthianapthen-2-one (9.63 g, quantitative crude yield) as a dark red solid.

¹HNMR (CDCl₃) δ 7.19 (t, J=7.8 Hz, 1H), 7.11 (d, J=6.8 Hz, 1H), 7.00 (d, J=7.3 Hz, 1H), 3.82 (s, 2H), 2.51 (q, J=7.8 Hz, 2H), 1.17 (t, J=7.8 Hz, 3H).

Preparation of 4-Ethylbenzo[b]thiophene.

To a solution of 4-ethylthianapthen-2-one (19.5 g, 110 mmol) in CH₂Cl₂ (1.15 L) was added dropwise 1.0 M diisobutylaluminum hydride in toluene (150 mL, 150 mmol) at 0° C. The solution was stirred at 0° C. for 2 h. The reaction was quenched with conc. HCl (700 mL) added dropwise over a period of 1.5 h. This mixture was then stirred vigorously for 2 h. The layers were separated, and the organic layer was washed with brine (1×500 mL), dried over MgSO₄ and concentrated. The residue was purified by medium pressure chromatography (100% hexanes) to give 4-ethylbenzo[b]thiophene as a yellow oil (6.37 g, 37%). EIMS 162 M⁺.

¹HNMR (CDCl₃) δ 7.53 (d, J=7.8 Hz, 1H), 7.09 (t, J 7.8 Hz, 1H), 7.05 (d, J=6.4 Hz, 1H), 6.98 (distorted d, 2H), 2.80 (q, J=7.8 Hz, 2H), 1.15 (t, J=7.8 Hz, 3H).

Preparation of N-t-Butoxycarbonyl-4-(4-ethylbenzo[b]thiophen-2-yl)-2-methyl-4-piperidinol.

To a solution of 4-ethylbenzo[b]thiophene (6.37 g, 39.2 mmol) in dry THF (200 mL) at −78° C. was added 1.6 M n-BuLi in hexanes (27.0 mL, 43.2 mmol). The solution was stirred at −78° C. for 2 h. N-t-Butoxycarbonyl-2-methyl-4-piperidone (6.70 g, 31.4 mmol) dissolved in THF (20 mL) was added via a cannula at −78° C. The reaction mixture was stirred at −78° C. for 3 h. The reaction was then quenched with 200 mL of saturated aqueous NH₄Cl solution. The mixture was extracted with EtOAc (1×200 mL). The combined organic layers were then dried over MgSO₄ and filtered. The filtrate was concentrated and purified by medium pressure chromatography (20% EtOAc/hexanes) to give the final title compound, N-t-butoxycarbonyl-4-(4-ethylbenzo[b]thiophen-2-yl)-2-methyl-4-piperidinol as a white foam (6.58 g, 56%). IR (CHCl₃) 3425 (br), 1664, 1692 cm⁻¹. Ion Spray MS 376 (M+H)⁺; 302 (M-73)⁺ (base peak); 434 (M+CH₃COO⁻)⁻. C₂₁H₂₉NO₃S

### EXAMPLE 1

Preparation of 4-(3-Methylbenzo[b]thiophen-5-yl)-piperidine hydrochloride.

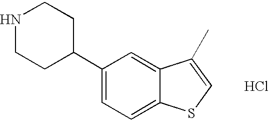

Scheme I, Step C: To a solution of 1-(t-butyloxycarbonyl)-4-(3-methyl-1-trimethylsilylbenzo[b]thiophen-5-yl)-piperidin-4-ol (458 g, 1.09 mol, from preparation 2) in CH₂Cl₂ (4.6 L) was added 871 mL (5.46 mol, 5.0 equiv) of triethylsilane. The mixture was cooled to −30° C. and 420 mL of trifluoroacetic acid (5.45 mol, 5.0 equiv) was added dropwise to the solution over 35 minutes. The mixture was stirred for 2.5 hours while gradually warming to 13° C. An additional 420 mL of trifluoroacetic acid was added over 15 minutes. After warming to room temperature over 3.5 hours, ice (6 L), water (5 L), and concentrated aqueous NaOH (628 mL, 12.0 mol, 11.0 eq) were added. The layers were separated and the aqueous layer was extracted with two 1.5 L portions of CH₂Cl₂. The organic layers were combined, dried (NaSO₄), and concentrated under vacuum to give a clear, colorless oil, which was redissolved in 4 L of ether. The hydrochloride salt was formed by dropwise addition of a solution of HCl in EtOAc (245 mL) until the slurry pH measured 2-3. The resulting slurry was stirred for 2 hours, filtered, rinsed with ether, and dried overnight in a vacuum oven at 45° C. to give 271 g of white crystalline 4-(3-methylbenzo[b]thiophen-5-yl)-piperidine hydrochloride (92.8% yield).

¹H NMR (500 MHz, DMSO) δ 2.10-2.20 (m, 2), 2.30 (q, 2), 2.42 (s, 3), 2.93 (m, 1), 3.0-3.10 (m, 2), 7.09 (s, 1), 7.25 (d, 1), 7.57 (s, 1), 7.80 (d, 1); ¹³C NMR (75 MHz, DMSO) δ 13.5, 29.6, 38.9, 43.4, 119.3, 122.7, 122.9, 123.2, 131.5, 137.7, 139.6, 140.9. Anal. Calcd for C₁₄H₁₈CINS: C, 62.79; H, 6.77; N, 5.23. Found: C, 62.66; H, 6.65; N, 5.24.

### EXAMPLE 2

Preparation of 1-(t-butyloxycarbonyl)-4-(3-Methylbenzo[b]thiophen-5-yl)-piperidine hydrochloride.

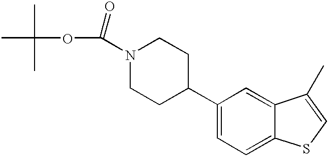

Scheme I, Step A: To a solution of 1-(t-butyloxycarbonyl)-4-(3-methyl-1-trimethylsilylbenzo[b]thiophen-5-yl)-piperidin-4-ol (458 g, 1.09 mol, from preparation 2) in CH₂Cl₂ (4.6 L) is added 871 mL (5.46 mol, 5.0 equiv) of triethylsilane. The mixture is cooled to −30° C. and 420 mL of trifluoroacetic acid (5.45 mol, 5.0 equiv) is added dropwise to the solution over 35 minutes. The mixture is stirred for 1 hour while gradually warming to 5° C. Then ice (6 L), water (5 L), and concentrated aqueous NaOH (314 mL, 6.0 mol, 5.5 eq) are added. The layers are separated and the aqueous layer is extracted with two 1.5 L portions of CH₂Cl₂. The organic layers are combined, dried (Na₂SO₄), and concentrated under vacuum to provide the title compound.

### EXAMPLE 3

Preparation of 4-(6-methoxybenzo[b]thiophen-2-yl)-2-methylpiperidine HCl.

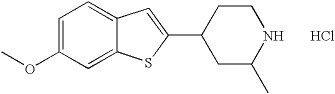

Scheme I, step C: The title compound is prepared from (±)N-t-butoxycarbonyl-4-hydroxy-4-(6-methoxybenzo[b]thiophen-2-yl)-2-methylpiperidine (prepared in preparation 3) in a manner analogous to the procedure described in Example 1.

### EXAMPLE 4

25 Preparation of (±) N-t-Butoxycarbonyl-4-(6-methoxybenzo[b]thiophen-2-vl)-2-methylpiperidine.

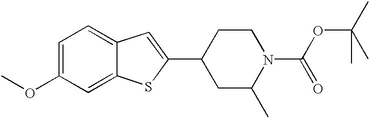

Scheme I, step A: The title compound is prepared from (±)N-t-butoxycarbonyl-4-hydroxy-4-(6-methoxybenzo[b]thiophen-2-yl)-2-methylpiperidine (prepared in preparation 3) in a manner analogous to the procedure described in Example 2.

### EXAMPLE 5

Preparation of 4-(6-methylbenzo[b]thiophen-2-yl)piperidine HCl.

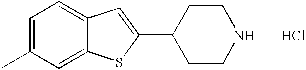

Scheme I, step C: The title compound is prepared from N-t-butoxycarbonyl-4-hydroxy-4-(6-methylbenzo[b]thiophen-2-yl)piperidine (prepared in preparation 4) in a manner analogous to the procedure described in Example 1.

### EXAMPLE 6

Preparation of N-t-Butoxycarbonyl-4-(6-methylbenzo[b]thiophen-2-yl)piperidine.

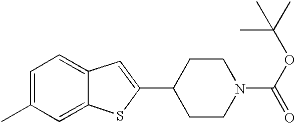

Scheme I, step A: The title compound is prepared from N-t-butoxycarbonyl-4-hydroxy4-(6-methylbenzo[b]thiophen-2-yl)piperidine (prepared in preparation 4) in a manner analogous to the procedure described in Example 2.

### EXAMPLE 7

Preparation of 2-methyl-4-(6-methylbenzo[b]thiophen-2-yl)piperidine HCl.

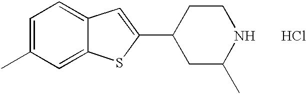

Scheme I, step C: The title compound is prepared from N-t-butoxycarbonyl-4-hydroxy-2-methyl-4-(6-methylbenzo[b]thiophen-2-yl)piperidine (prepared in preparation 5) in a manner analogous to the procedure described in Example 1.

### EXAMPLE 8

Preparation of N-t-Butoxycarbonyl-2-methyl-4-(6-methylbenzo[b]thiophen-2-yl)piperidine.

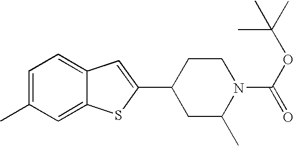

Scheme I, step A: The title compound is prepared from N-t-butoxycarbonyl-4-hydroxy-2-methyl-4-(6-methylbenzo[b]thiophen-2-yl)piperidine (prepared in preparation 5) in a manner analogous to the procedure described in

### Example 2

### EXAMPLE 9

Preparation of 4-(8-methoxynaphth-2-yl)piperidine HCl.

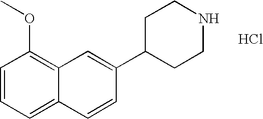

Scheme I, step C: The title compound is prepared from N-t-butoxycarbonyl-4-(8-methoxynaphth-2-yl)-4-piperidinol (prepared in preparation 6) in a manner analogous to the procedure described in Example 1.

### EXAMPLE 10

Preparation of N-t-Butoxycarbonyl-4-(8-methoxynaphth-2-vl)piperidine.

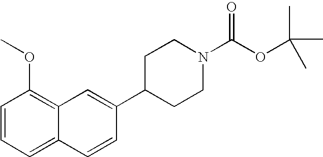

Scheme I, step A: The title compound is prepared from N-t-butoxycarbonyl-4-(8-methoxynaphth-2-yl)-4-piperidinol (prepared in preparation 6) in a manner analogous to the procedure described in Example 2.

### EXAMPLE 11

Preparation of 4-(6-methoxynaphth-2-yl)-2-methylpiperidine HCl.

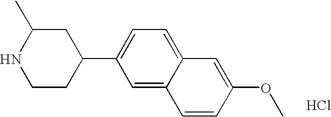

Scheme I, step C: The title compound is prepared from 1-(t-butyloxycarbonyl)-4-(6-methoxynaphth-2-yl)-2-methylpiperidin-4-ol (prepared in preparation 7) in a manner analogous to the procedure described in Example 1.

### EXAMPLE 12

Preparation of 1-(t-Butyloxycarbonyl)-4-(6-methoxynaphth-2-vl)-2-methylpiperidine.

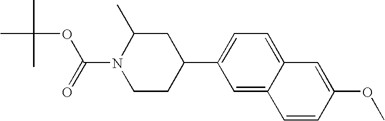

Scheme I, step A: The title compound is prepared from 1-(t-butyloxycarbonyl)-4-(6-methoxynaphth-2-yl)-2-methylpiperidin-4-ol (prepared in preparation 7) in a manner analogous to the procedure described in Example 2.

### EXAMPLE 13

Preparation of 4-(4-ethylbenzo[b]thiophen-2-yl)-2-methylpiperidine HCl.

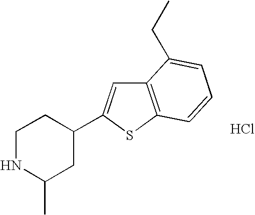

Scheme I, step C: The title compound is prepared from N-t-butoxycarbony-4-(4-ethylbenzo[b]thiophen-2-yl)-2-methyl-4-piperidinol (prepared in preparation 8) in a manner analogous to the procedure described in Example 1.

### EXAMPLE 14

Preparation of N-t-Butoxycarbonyl-4-(4-ethylbenzo[b]thiophen-2-vl)-2-methylpiperidine.

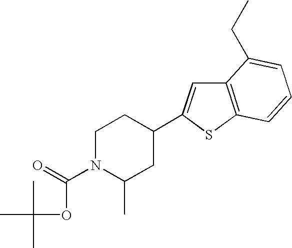

Scheme I, step A: The title compound is prepared from N-t-butoxycarbonyl-4-(4-ethylbenzo[b]thiophen-2-yl)-2-methyl-4-piperidinol (prepared in preparation 8) in a manner analogous to the procedure described in Example 2.

### EXAMPLE 15

Preparation of 4-(4-Methoxybenzo[b]thiophen-2-yl)-piperidine HCl.

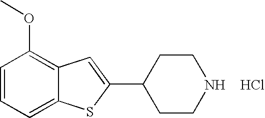

Scheme I, step C: The title compound was prepared from N-t-butoxycarbonyl-4-(4-methoxybenzo[b]thiophen-2-yl)-4-piperidinol in a manner analogous to the procedure described in Example 1.

### EXAMPLE 16

Preparation of N-t-Butoxycarbonyl-4-(4-methoxybenzo[b]thiophen-2-yl)-piperidine.

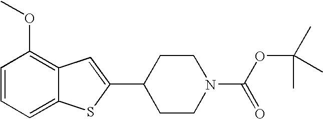

Scheme I, step A: The title compound is prepared from N-t-butoxycarbonyl-4-hydroxy-4-(4-methoxybenzo[b]thiophen-2-yl)-piperidine in a manner analogous to the procedure described in Example 2.

We claim:

1. A process for preparing a reduced N-protected amine compound comprising reducing an N-protected amine possessing a secondary or tertiary alcohol with triethylsilane and trifluoroacetic acid.

2. The process according to claim 1 wherein the N-protected amine is an N-protected-4-substituted piperidine possessing a tertiary alcohol at the 4-position of the piperidine ring.

3. The process according to claim 2 wherein the N-protecting group on the amine is an acid labile nitrogen protecting group.

4. The process according to claim 3 wherein the acid labile nitrogen protecting group is a t-butyloxycarbonyl.

5. The process according to claim 4 wherein the piperidine has a heterocycle, substituted heterocycle, substituted alkenyl or substituted aryl at the 4-position wherein the substituted heterocycle, substituted alkenyl or substituted aryl are substituted with from 1 to 3 suitable activating groups.

6. A process for preparing a compound of formula I: 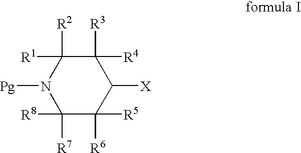
wherein Pg represents a suitable nitrogen protecting group;
X represents a heterocycle, substituted heterocycle, substituted alkenyl or substituted aryl wherein the substituted heterocycle, substituted alkenyl or substituted aryl are substituted with from 1 to 3 suitable activating groups; and
R¹, R², R³, R⁴, R⁵, R⁶, R⁷, and R⁸ each independently represent an alkyl, alkenyl, cycloalkyl, aryl, substituted aryl, heterocycle, or substituted heterocycle, comprising treating a compound of formula II: 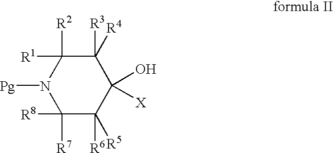
wherein the substituents are defined as above, with triethylsilane and trifluoroacetic acid.

7. The process according to claim 6 wherein Pg is an acid labile nitrogen protecting group.

8. The process according to claim 7 wherein the acid labile nitrogen protecting group is a t-butoxycarbonyl.

9. The process according to claim 8 wherein R¹, R², R³, R⁴, R⁵, R⁶, R⁷, and R⁸ each independently represent hydrogen or C₁-C₄ alkyl.

10. The process according to claim 9 wherein R¹, R², R³, R⁴, R⁵, R⁶, R⁷, and R⁸ each independently represent hydrogen or methyl.

11. A process for preparing a compound of formula Ia: 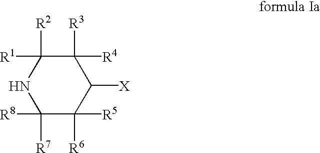
wherein X represents a heterocycle, substituted heterocycle, substituted alkenyl or substituted aryl wherein the substituted heterocycle, substituted alkenyl or substituted aryl are substituted with from 1 to 3 suitable activating groups; and
R¹, R², R³, R⁴, R⁵, R⁶, R⁷, and R⁸ each independently represent an alkyl, alkenyl, cycloalkyl, aryl, substituted aryl, heterocycle, or substituted heterocycle, comprising treating a compound of formula II: 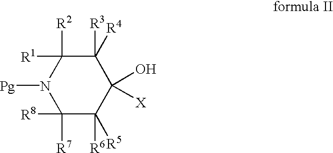
wherein Pg represents a suitable nitrogen protecting group and the remaining substituents are defined as above, with triethylsilane and excess trifluoroacetic acid.
